SEQUENCE LISTING (1 sequence)

SEQ ID NO: 1 - protein, 42 aa, human [sequence omitted]

(19) United States
(12) Patent Application Publication
(10) Pub. No.: US 2021/0285969 A1
(43) Pub. Date: Sep. 16, 2021

(54) METHODS FOR DIAGNOSING A CEREBRAL AMYLOID ANGIOPATHY

(71) Applicants: Sorbonne Universite, Paris (FR); INSERM (Institut National De La Sante Et De La Recherche Medicale), Paris (FR); Assistance Publique - Hopitaux De Paris, Paris (FR)

(72) Inventors: Pierre Aucouturier, Montreuil (FR); Yannick Chantran, Paris (FR); Jean Capron, Paris (FR); Sonia Alamowitch, Paris (FR)

(21) Appl. No.: 17/263,063

(22) PCT Filed: Jul. 23, 2019
(86) PCT No.: PCT/EP2019/069836

§ 371 Date: Jan. 25, 2021

(30) Foreign Application Priority Data
Jul. 23, 2018 (EP) 18306003.7

(51) Int. Cl. G01N 33/68

(52) U.S. Cl. CPC G01N 33/6896; G01N 2333/4709; G01N 33/6854

(57) ABSTRACT

The invention provides an in vitro method for diagnosing cerebral amyloid angiopathy (CAA), or for diagnosing or determining the risk of developing a complication of CAA, in a human subject, which method comprises analyzing serial dilutions of a plasma or serum sample of the subject, for determining at least one binding parameter of antibodies present therein, wherein said antibodies are anti-Aβ amyloid peptide(s) antibodies.

The present invention relates to methods for diagnosing or determining the risk of developing clinical manifestations of cerebral amyloid angiopathy (CAA) in a human subject, which is frequent in elderly.

### BACKGROUND OF THE INVENTION

Cerebral amyloid angiopathy results from deposits of amyloid material inside blood vessel walls of the cortex and/or leptomeninges. The prevailing form of sporadic CAA is due to progressive, age-dependent accumulation of aggregated amyloid-β peptide (Aβ), mainly the 40-aminoacid form (Aβ₁₋₄₀). The incidence of CAA is elevated in the elderly (over 35%, 45% and 70% during the 6^{th}, 7^{th} and 8^{th} decades, respectively) and even higher in patients with Alzheimer's disease (AD). Lobar hemorrhage (LH) is the main complication of sporadic CAA and a major health concern due to frequent short-term mortality and growing incidence linked to increasing prescription of antithrombotic drugs in aged people (Béjot et al., 2013). Besides hemorrhagic features (CAA-he), CAA-related inflammation (CAA-ri) is a rare but severe complication that manifests as a corticosensitive Aβ-related angiitis (ABRA) and/or perivascular cerebral inflammation (Salvarani et al., 2013).

Pathophysiological mechanisms triggering CAA-he and CAA-ri manifestations remain obscure. However, it is worth noting that AD patients receiving monoclonal anti-Aβ antibody infusions frequently develop dose-dependent severe adverse events, in connection with amyloid-related imaging abnormalities (ARIA) analogous to magnetic resonance imaging (MRI) features of CAA-he (ARIA-H for hemorrhagic) and CAA-ri (ARIA-E for effusion) (Sperling et al., 2011). Occurrence of ARIA varies in both form and frequency as a function of the type of used anti-Aβ antibody (review in Chantran et al., 2019).

Definite pathological diagnosis of CAA is either post-mortem or requires invasive cerebral biopsy, and therefore it is seldom achieved. At present, neuroimaging markers are the basic tools for identifying probable CAA on the basis of the Boston criteria (Knudsen et al., 2001; Linn et al., 2010). There is thus an acute need for early diagnosis of CAA with circulating biomarkers that would allow discriminating or predicting the occurrence of CAA-he and CAA-ri. Especially, early diagnosis of CAA-he may allow prevention, including avoidance of thrombolytic and anti-thrombotic therapies (Wilson et al 2018). Indeed a particular issue is presently the screening for asymptomatic CAA in patients who are being prescribed long term anticoagulation, for instance in atrial fibrillation. Asymptomatic CAA causes a higher risk of iatrogenic cerebral hemorrhage (Wilson et al. 2018). The only reliable test to date is cerebral MRI, which cannot be performed as a screening test in such a large population of patients. The availability of a blood biomarker would stratify subjects who need an MRI.

A blood biomarker of CAA-ri may shorten what remains a challenging diagnosis, prevent cerebral biopsy and therapeutic testing with potentially iatrogenic immunosuppressant drugs, and provide a biomarker of disease progression under treatment, thus reducing the high mortality rate of this disease.

### SUMMARY OF THE INVENTION

The invention provides an in vitro method for diagnosing cerebral amyloid angiopathy (CAA), or for diagnosing or determining the risk of developing a complication of CAA, in a human subject, which method comprises analyzing serial dilutions of a plasma or serum sample of the subject, for determining at least one binding parameter of antibodies present therein, wherein said antibodies are anti-Aβ amyloid peptide(s) antibodies.

Preferably the method distinguishes the class and/or subclass of such antibodies.

In preferred embodiments, the complication may be hemorrhagic CAA or inflammatory CAA.

In preferred embodiments, the Aβ peptide is Aβ₁₋₄₀ or Aβ₁₋₄₂ peptide.

In a particular embodiment, the Aβ peptide is soluble Aβ₁₋₄₀ and the anti-soluble Aβ₁₋₄₀ antibodies to monitor preferably are IgG antibodies, preferably IgG3, IgG1 or IgG4 antibodies, still preferably IgG3 or IgG4 antibodies.

In another embodiment, the Aβ peptide is fibrillar Aβ₁₋₄₀, and the anti-fibrillar Aβ₁₋₄₀ antibodies preferably are IgM or IgA antibodies.

In a particular embodiment, the antibodies are detected by means of a chromogenic or fluorescent labelling, preferably the antibodies are detected by means of an indirect immunoassay on immobilized antigen using a secondary anti-human immunoglobulin antibody that carries a chromogenic enzyme or fluorescent label.

The binding parameter may typically be i) the titer of said antibodies as determined at 50% maximum binding, ii) the affinity or avidity constant, iii) the steepness of the dilution curve, and/or iv) the maximum optical density observed, preferably normalized with an internal standard.

In a most preferred embodiment, the method comprises determining an index (designated “MAST-index”) that is a weighted summation of the following binding parameters: i) the titer of said antibodies as determined at 50% maximum binding, ii) the affinity or avidity constant, iii) the maximum optical density, preferably normalized with an internal standard, and iv) the steepness of the dilution curve.

The method may particularly comprise determining the MAST-index of anti-soluble Aβ₁₋₄₀ IgG antibodies, wherein a higher MAST-index of anti-soluble Aβ₁₋₄₀ IgG antibodies compared to control is indicative of a subject with a CAA complication of the inflammatory (CAA-ri) type, or of a higher risk for the subject to develop a CAA-ri, preferably wherein the anti-soluble Aβ₁₋₄₀ IgG antibodies are anti-soluble Aβ₁₋₄₀ IgG3 or IgG4 antibodies.

The method may also, or alternatively, comprise determining the MAST-index of anti-fibrillar Aβ₁₋₄₀ IgM or IgA antibodies, wherein a higher MAST-index of anti-fibrillar Aβ₁₋₄₀ IgM or IgA antibodies compared to control is indicative of a subject with a CAA complication of the haemorrhagic (CAA-he) type, or of a higher risk for the subject to develop a CAA-he.

In a particular embodiment the method comprises determining at least one binding parameter of anti-soluble Aβ₁₋₄₀ IgG antibodies and at least one binding parameter of anti-fibrillar Aβ₁₋₄₀ IgM antibodies in a plasma or serum sample of the subject.

Such method may comprise conducting a multiplex immunoassay.

The method of the invention makes it possible to detect and monitor cerebral amyloid angiopathy manifestations that are either spontaneous or induced by anti-Aβ immunotherapy.

It further helps stratifying subjects who require an MRI.

The invention further provides an in vitro method for diagnosing cerebral amyloid angiopathy (CAA), or for diagnosing or determining the risk of developing a complication of CAA, especially a CAA complication of the inflammatory (CAA-ri) type, in a human subject, which method comprises measuring the anti-soluble Aβ₁₋₄₀ IgG3 or IgG4 antibodies.

### LEGENDS TO THE FIGURES

FIG. 1. MRI findings of hemorrhagic features of CAA. A, cerebral MRI in magnetic susceptibility sequence (T2*) shows acute right frontal lobar hemorrhage, a left parietal lobar microbleed and diffuse cortical superficial siderosis. B, cerebral MRI in fluid attenuation sequence (FLAIR) shows the lobar hemorrhage but the microbleed and the superficial siderosis are not visible on that MRI sequence.

FIG. 2. Clinical and MRI findings of an illustrative case of CAA-related inflammation. A 81-year-old patient (patient no. 1), presents with subacute onset of confusion and progressive aphasia over 2 months. A) cerebral MRI in fluid attenuation sequence (FLAIR) shows left temporo-occipital hypersignal responsible for a mass effect. B) cerebral MRI in magnetic susceptibility sequence (T2*) shows multiple bilateral lobar cortical microhemorrhages. C) cerebral MRI in FLAIR sequence shows hypersignal regression after 6 weeks of corticosteroid therapy associated with clinical improvement.

FIG. 3. Determination of dilution curve parameters by sigmoid modeling and linearization procedure. The left-side panel illustrates the dilution curve obtained from a human serum sample following acidic dissociation of circulating immune complexes and neutralization, incubated on coated soluble Aβ1-42 (s42) antigen and revealed with horseradish peroxidase (HRP)-conjugated anti-IgG secondary antibody. The dashed thick line represents the sigmoid modeling of the curve, accurately described by i) the y value of the left-sided plateau (Max), which reflects the antigen recognition diversity; ii) the x value at the inflexion point (Titer), which depends in part on the concentration of antibodies in the sample; iii) the steepness at the inflexion point (Steepness), which reflects cooperativity phenomena involved in the binding of the antibody. The right-side panel shows the linearization of the same experimental points and sigmoid model, and allows the determination of an apparent constant of avidity (K′a App), which reflects the mean affinity of the diverse antibody binding sites.

FIG. 4. A. circulating anti-fibrillar Aβ1-40 IgM MAST-index in the 3 study groups; B, ROC curve of anti-fibrillar Aβ1-40 IgM MAST-index for discriminating CAA-he from controls. Boxes indicate IQR. Central bars indicate median. Error bars indicate last extreme value below 1.5 times the 2nd or 3rd quartile. AU, arbitrary units. CAA-he, hemorrhagic acute complication of cerebral amyloid angiopathy. CAA-ri, cerebral amyloid angiopathy-related inflammation. Se, sensitivity. Sp, specificity. AUC, area under the curve.

FIG. 5. Circulating anti-soluble Aβ1-40 IgG3 MAST-index in the 3 study groups. Boxes indicate IQR. Central bars indicate median. Error bars indicate last extreme value below 1.5 times the 2nd or 3rd quartile. CAA-he, hemorrhagic acute complication of cerebral amyloid angiopathy. CAA-ri, cerebral amyloid angiopathy-related inflammation. *: p-value<0.05; **: p-value<0.01; ***: p-value<0.001.

FIG. 6. Circulating anti-soluble Aβ1-40 IgG4 MAST-index in the 3 study groups. Boxes indicate IQR. Central bars indicate median. Error bars indicate last extreme value below 1.5 times the 2nd or 3rd quartile. CAA-he, hemorrhagic acute complication of cerebral amyloid angiopathy. CAA-ri, cerebral amyloid angiopathy-related inflammation. *: p-value<0.05; **: p-value<0.01; ***: p-value<0.001.

FIG. 7. Circulating anti-fibrillar Aβ1-40 IgA MAST-index in the 3 study groups. Boxes indicate IQR. Central bars indicate median. Error bars indicate last extreme value below 1.5 times the 2nd or 3rd quartile. CAA-he, hemorrhagic acute complication of cerebral amyloid angiopathy. CAA-ri, cerebral amyloid angiopathy-related inflammation. *: p-value<0.05; **: p-value<0.01; ***: p-value<0.001.

FIG. 8. ROC curve evaluating the performances of anti-soluble Aβ1-40 IgG3 MAST-index to discriminate CAA-he patients from age-matched controls.

FIG. 9. ROC curve evaluating the performances of anti-soluble Aβ1-40 IgG3 MAST-index to discriminate CAA-ri patients from age-matched controls.

FIG. 10. ROC curve evaluating the performances of anti-soluble Aβ1-40 IgG3 MAST-index to discriminate CAA patients with acute presentation from age-matched controls.

FIG. 11. ROC curve evaluating the performances of anti-soluble Aβ1-40 IgG4 MAST-index to discriminate CAA-he patients from age-matched controls.

FIG. 12. ROC curve evaluating the performances of anti-soluble Aβ1-40 IgG4 MAST-index to discriminate CAA-ri patients from age-matched controls.

FIG. 13. ROC curve evaluating the performances of anti-soluble Aβ1-40 IgG4 MAST-index to discriminate CAA patients with acute presentation from age-matched controls.

FIG. 14. ROC curve evaluating the performances of anti-fibrillar Aβ₁₋₄₀ IgA MAST-index to discriminate CAA-he patients from age-matched controls.

### DETAILED DESCRIPTION OF THE INVENTION

### Definitions

The subject may be any human patient, regardless of the gender or age, suspected of having a cerebral amyloid angiopathy or being at risk of, or predisposed to, developing cerebral amyloid angiopathy. In a particular embodiment, the subject is more than 50 years old. In a particular embodiment, the subject may be afflicted with Alzheimer's disease (AD). Asymptomatic subjects are included.

Subjects at risk of developing a cerebral amyloid angiopathy include every subject over 50 years old, patients diagnosed with typical sporadic AD, ‘atypical’ sporadic focal forms of AD such as posterior cortical atrophy and primary progressive aphasia, patients with hereditary forms of AD, patients with Down's syndrome as well as patients treated with anti-Aβ immunotherapy. Other risk factors include aging, genetic risk factors, such as mutations of the amyloid precursor protein (APP) or presenilin genes, or the ε2 or ε4 alleles of the ApoE gene, or non-genetic risk factors, such as hypertension, or thrombolytic, anticoagulation, and antiplatelet therapies.

Patients who are being prescribed long term anticoagulation therapy, for instance in atrial fibrillation, are particular candidates for the present test.

The amyloid beta peptide (Aβ peptide) may be considered the main product of the proteolytic processing of the Amyloid Precursor Protein (APP). There are various isoforms of Aβ that differ by the number of amino acid residues at the C-terminal end of the peptide. The iso form Aβ40 has 40 residues. Aβ42 is less soluble in water saline buffers than Aβ40 and is more prone to aggregation. The Aβ peptides have a specific type of β-sheet arrangement that favors the polymerization and aggregation, leading to the formation of oligomeric species that diffuse through the interstitial fluids. Aβ monomers tend to aggregate and polymerize, forming oligomers, protofibrils and fibrils.

The soluble or fibrillar material can be characterized by several methods, e.g. direct observation of fibrils, oligomers or monomers by Transmission Electron Microscopy or Atomic Force Microscopy; absence of fluorescence (for soluble non-amyloid monomers or oligomers) or presence of fluorescence (for amyloid fibrils) after incubation with Thioflavine derivatives.

The term “soluble” means the ability for a given substance, the solute (an example in the instant invention is the Aβ oligomer) to dissolve in a solvent. Within the context of the instant invention, soluble Aβ peptides are capable of being fractionated by centrifugation.

The term “fibrillar” means that the Aβ peptides and oligomeric complexes are aligned in a morphologically distinct pattern known as amyloid protofibrils or amyloid fibrils.

An anti-Aβ immunotherapy typically refers either to passive immunization by injecting monoclonal antibodies (mAb) directed against the Aβ peptide, e.g. bapineuzumab, ponezumab, solanezumab, gantenerumab, aducanumab, crenezumab or BAN-2401, or to active immunization by using antigens generating a B-cell elective response without anti-Aβ T-cell response, thus generating solely anti-Aβ antibodies, e.g. CAD-106.

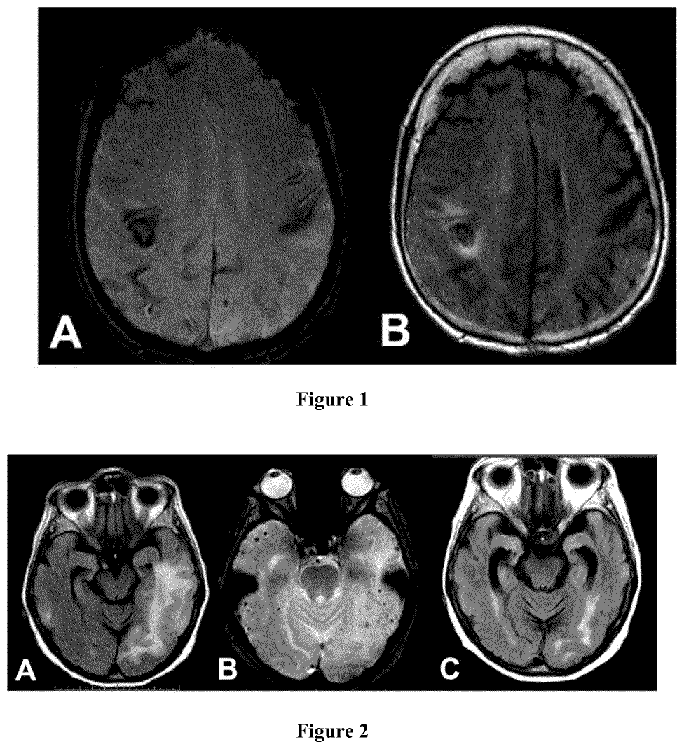

The term “CAA-he” refers to a hemorrhagic acute complication of cerebral amyloid angiopathy. FIG. 1 shows MRI findings of hemorrhagic features of CAA.

“CAA-ri” means cerebral amyloid angiopathy-related inflammation. It manifests as a corticosensitive Aβ-related angiitis (ABRA) and/or perivascular cerebral inflammation. CAA-ri manifests as acute or subacute symptoms with headache, decrease in consciousness, behavioral change, or focal neurological signs and seizures. MRI shows unifocal or multifocal, corticosubcortical or deep white matter hyperintensities (WMH) lesions that are asymmetric and extend to the immediately subcortical white matter, associated to corticosubcortical hemorrhagic lesions characteristic of CAA (FIG. 2).

The biological sample is derived from blood. It may be any plasma or serum sample. The biological sample can be used directly, or it can be subjected to a processing step before being tested.

The term “control” (or “control value”) typically refers to a value yielded upon analysis of a serum from a healthy individual (“control subject”) who is not affected with CAA-he nor CAA-ri as assessed using clinical and imaging studies, as described below. These control values represent reference data for comparing CAA-he and CAA-ri values.

In order to evaluate the progression of the pathological condition, it may be useful to test a patient and to verify the effect of a treatment or the progression of the pathological condition by testing the patient again, for example with a gap of several months. In this case, the results of the second test are compared with the results of the first test.

In the context of the present invention, a “binding parameter” is a quantitative figure that can be determined upon measurement of antibody binding to immobilized Aβ peptide using serial dilutions of a given serum or plasma sample. Four distinct binding parameters may typically be defined, i.e. i) the titer of said antibodies as determined at 50% maximum binding, ii) the affinity or avidity constant, iii) the steepness of the dilution curve, and/or iv) the maximum optical density observed, preferably normalized with an internal standard. Such parameters are described in the Experimental section as well. A “MAST Index” is herein defined that can be calculated as described in the Experimental section. It is a sum of

a1*(the maximum optical density observed)+a2*(the affinity or avidity constant)+a3*(the steepness of the dilution curve)+a4*(the titer of said antibodies as determined at 50% of maximum binding).

The coefficients a1, a2, a3, a4 are preferably defined to weigh on the parameters as follows:

a1=5 to 30% of the sum of all coefficients (a1+a2+a3+a4)
a2=25 to 50% of the sum of all coefficients (a1+a2+a3+a4)
a3=0 to 20% of the sum of all coefficients (a1+a2+a3+a4)
a4=25 to 50% of the sum of all coefficients (a1+a2+a3+a4). In a particular embodiment, the MAST Index is calculated as Index=0.2316*Max+0.6717*Titer+0.1413*Steepness+0.6894*K′A App, it being understood that the coefficients may be proportionally modified.

Epitope diversity can be measured by any method capable of measuring the amount of antibody bound to the capture antigen, under conditions where antibodies from the sample are present in excess regarding to amount of capture antigen present.

Antibody concentration can be measured by any method capable of measuring the amount of antibody bound to the capture antigen, under conditions where the amount of capture antigen present is in excess regarding to amount of antibodies in the sample, and preferably under conditions where half the amount of capture antigen is bound to antibodies (giving half the signal obtained for Average epitope diversity measure).

Avidity can be measured by any method capable of measuring the apparent KD constant as defined as ([Free capture Ag]*[Free antibody])/[Bound antibody] in equilibrium conditions.

The term “capture antigen” is intended to mean an antigen, preferably attached to a solid phase, which is capable of retaining said at least one antibody present in a biological sample, by affinity binding. The capture antigen may be labeled. In the present invention, an example of capture antigen is a Aβ peptide, such as a Aβ40 peptide, a Aβ42, or fragments thereof, e.g a Aβ peptide of 38 or 39 or 41 amino acids only. More generally, as a capture antigen, the methods of the invention may typically make use of a peptide or modified peptide that includes a substantial part of the sequence of the 42 residues of the human amyloid beta peptide (DAEFRHDSGYEVHHQKLVFFAEDVGSNKGAIIGLMVGGVVIA). Modified peptides include single aminoacid substitution or modification (e.g. pyroglutamyl-Aβ), cross-linking, or covalent binding with another molecule, such as carbohydrates or phosphate.

The term “labeled” refers both to a direct labeling (by means of enzymes, radioisotopes, fluorochromes, luminescent compounds, etc.) and to an indirect labeling (for example by means of antibodies which are themselves directly labeled or using reagents of a labeled “affinity pair”, such as, but non exclusively, the labeled avidin-biotin pair, etc.).

Assaying the Auto-Antibodies

The methods of the invention encompass analyzing biological samples that may contain circulating auto-antibodies that recognize several possible forms of Aβ peptides, including soluble Aβ₁₋₄₀ peptide or fibrillar Aβ₁₋₄₀ peptide, or soluble Aβ₁₋₄₂ peptide or fibrillar Aβ₁₋₄₂ peptide.

In preferred embodiments, the methods of the invention comprise determining the MAST-index of anti-soluble Aβ₁₋₄₀ IgG3 antibodies or anti-fibrillar Aβ₁₋₄₀ IgM antibodies in a plasma or serum sample of the subject.

The biological sample is a plasma or serum sample, preferably a serum sample, at several dilutions, preferably between 1/20th and 1/50000th, in order to obtain a dilution curve.

Determination of the dilution curve parameters and the determination of the MAST Index, as defined herein, can be achieved as described in greater details below (see Experimental Section).

Advantageously, the binding parameters can be determined by an immunoassay. The biological sample can be optionally treated in a prior step, or brought directly into contact with at least one capture antigen. The method according to the invention can be carried out according to various formats well known to those skilled in the art: in solid phase or in homogeneous phase; in one step or in two steps, or more; in a competition method, by way of nonlimiting examples.

According to one preferred embodiment, the capture antigen is immobilized on a solid phase. By way of nonlimiting examples of a solid phase, use may be made of microplates, in particular polystyrene microplates. Use may also be made of solid particles or beads, paramagnetic beads, or else polystyrene or polypropylene test tubes, etc.

An immunoassay format for detecting antibodies by competition is also possible. Other immunoassay modes can also be envisioned and are well known to those skilled in the art. ELISA assays, radioimmunoassays, immunofluorimetric assays, or any other detection technique can be used for revealing the presence of the antigen-antibody complexes formed.

The soluble or fibrillar Aβ peptides used as capture antigens may be prepared by any method known in the art, e.g. as described in Stine et al, 2011, or using the protocol described in greater details in the Experimental section below. Typically a synthetic Aβ peptide is dissolved in 1,1,1,3,3,3-hexafluoro-2-propanol (HFIP), HFIP is evaporated, and the dry peptide may be stored at −20° C. The peptide is resuspended in dimethylsulfoxide (DMSO). For soluble conditions, a buffer using physiologic concentration of salts and/or physiologic pH is added, while, for fibrillar conditions, acidic pH and/or low salt concentrations are required.

According to one particular preferred embodiment, the capture antigen is a soluble Aβ₁₋₄₀ peptide or a fibrillar Aβ₁₋₄₀ peptide.

By way of illustration, Aβ peptides, such as soluble or fibrillar Aβ₁₋₄₀ can be used as antigens in an indirect immunoassay such as an indirect ELISA. Serum samples can be pretreated for acidic dissociation of immune complexes prior to incubation in plates sensitized with each Aβ antigenic preparation. Bound antibodies belonging to IgM class, IgA class, and IgG class and subclasses are revealed using appropriate specific antibody reagents. For instance, conjugated anti-mouse IgG for monoclonal mouse anti-human IgG subclasses, or anti-human IgG, IgA, and IgM, preferably conjugated with peroxidase or alkaline phosphatase, may be used.

In another example, the capture antigen may be labelled, e.g. by being coupled to a glutathione S transferase (GST), before being deposited on a microplate.

In a particular embodiment, the method comprises determining both the MAST-index of anti-soluble Aβ₁₋₄₀ IgG3 antibodies and the MAST-index of anti-fibrillar Aβ₁₋₄₀ IgM antibodies in a blood or serum sample of the subject. In that embodiment, the method can comprise conducting a multiplex immunoassay, namely combining detection of anti-soluble β₁₋₄₀ IgG3 antibodies and detection of anti-fibrillar Aβ₁₋₄₀ IgM antibodies in a single reaction volume.

The diagnosis methods described herein allows prevention of CAA manifestations, more particularly CAA-he or CAA-ri, e.g. by avoiding additional risk factor(s) such as anti-Aβ immunotherapy, hypertension, or thrombolytic, anticoagulation, and antiplatelet therapies.

An immunosuppressive treatment could also be envisioned, as subacute leukoencephalopathy associated with CAA-related inflammation or angiitis was reported to respond.

Evaluation of the Efficacy of a Treatment

Another aspect of the invention is an in vitro method for evaluating the efficacy of a treatment for CAA manifestations, which comprises determining the presence and/or the amount of at least one antibody as defined above in a biological sample originating from a patient, at various times before, during or after the treatment, a decrease in the amount of said at least one antibody over time being indicative of an improvement in the CAA complications.

The Figures and Example below illustrate the invention without limiting its scope.

### Example: Distinctive Serum Anti-Aβ Antibodies Features During Haemorrhagic and Inflammatory Complications of Cerebral Amyloid Angiopathy

Material & Methods

Study Design and Participants

All patients and control subjects underwent a complete set of clinical, biological and imaging analyses. Medical files and MRI from 9 centers were all reviewed by one trained neurologist (JC) to insure that diagnosis criteria were evenly met. Diagnosis of CAA-he was made according to the revised Boston's criteria (Linn et al., 2010). Since controversy exists about the diagnostic value of microbleeds (MB) in the absence of LH (Martinez-Ramirez et al., 2015), a history of LH or a high number of MB (>50) were required to insure a diagnosis of CAA. Diagnosis of CAA-ri was made according to Auriel et al. (Auriel et al., 2016). Control subjects were selected on the following criteria: age >60 years; clinically: transient ischemic attack, seizure or differential diagnoses (vertigo or hypotension for instance), and absence of known cognitive deficit; on MRI: absence of spontaneous hemorrhage, MB or cSS, and absence of acute cerebral lesion.

MB count was rated according to the microbleed anatomical rating scale (MARS) (Gregoire et al., 2009). Leukoencephalopathy was graded according to the Fazekas's scale (Fazekas et al., 1987). Hippocampal atrophy was graded according to the Scheltens' criteria (Scheltens et al., 1992). Blood samples were collected in the acute phase, prior to corticosteroid administration for CAA-ri. Serum aliquots were kept frozen at −80° C. until use. The study protocol was approved by the Ethic Committee “Paris Ile de France V”.

TABLE 1
Characteristics of the Study Groups

| Clinical group | Control | CAA-he | CAA-ri |
|---|---|---|---|
| Number of subjects | 28 | 20 | 12 |
| Age, median (range), years | 71 (62-89) | 74 (60-90) | 72.5 (64-86) |
| Male/Female | 15/13 | 11/9 | 5/7 |
| Serum storing time, median | 19 (3-42) | 16 (3-50) | 27 (4-55) |
| (range), months | | | |

Abbreviations:
CAA-he, cerebral amyloid angiopathy-related hemorrhage;
CAA-ri, cerebral amyloid angiopathy-related inflammation.

Aβ Preparations

This protocol has been extensively described elsewhere (Charidimou et al., 2017; Yamada et al., 2015). Purified (>95%) synthetic Aβ₁₋₄₀ and Aβ₁₋₄₂ peptides (Proteogenix, Schiltigheim, France) were dissolved in 1,1,1,3,3,3-hexafluoro-2-propanol (HFIP; Sigma-Aldrich), and 150 or 450 μg aliquots were transferred to low retention tubes. Aliquots were evaporated, dried in a SpeedVac, and stored at −20° C. until use. For peptide soluble preparations, aliquots of lyophilized Aβ were dissolved in 104 dimethylsulfoxide (DMSO; Sigma-Aldrich), sonicated for 3 min at 300 Watts, mixed just before use with 90 μL of HEPES 30 mM NaCl 10 mM Cu²⁺10 eq pH 7.4 buffer, or HEPES 30 mM NaCl 160 mM Cu²⁺10 eq pH 7.4 buffer, respectively for Aβ₁₋₄₂ and Aβ₁₋₄₀. For fibril preparations, lyophilized Aβ was dissolved in 104 DMSO, sonicated 3 min at 300 Watts, mixed with 90 μL HCl 0.01N or HEPES 30 mM NaCl 160 mM pH 7.4 buffer, and incubated at 37° C. during 72 h or 15 days, respectively for Aβ₁₋₄₂ and Aβ₁₋₄₀.

Anti-Aβ Antibodies Detection by Multiplex ELISA

Freshly prepared soluble or fibrillar Aβ₁₋₄₀ and Aβ₁₋₄₂ (hereafter termed s40, s42, f40 or f42, respectively) were used as antigens in an indirect ELISA.

Freshly prepared soluble or fibrillar Aβ₁₋₄₀ and Aβ₁₋₄₂ (hereafter termed s40, s42, f40 or f42) were diluted to 15 μg/mL in coating buffer (HEPES 30 mM NaCl 160 mM or 10 mM (for Aβ₁₋₄₀ and Aβ₁₋₄₂, respectively) Cu2+10 eq (for monomers) pH 7.4), distributed at 100 μL per well into flat-bottomed ELISA plates (Greiner BioOne) and incubated 16 hours at 4° C. Blank control wells were filled with same volumes of corresponding buffer.

Serial dilutions of serum samples at 1:50 to 1:12800 in 0.1 M Glycine-HCl buffer pH 3.0, were left 40 minutes at 20° C. for dissociation of immune complexes, neutralized to pH 7.4 by adding the same volume of 2×PBS BSA 4% NaOH 0.02N, then 1004 were immediately deposited into s40-, s42-, f40- or f42-coated ELISA plates and incubated 1 h at 20° C. After 8 washes with PBS Tween-20 0.05%, bound antibodies of each IgG subclass were detected by 16 h incubation at 4° C. of monoclonal anti-human IgG1, IgG2, IgG3, or IgG4 antibodies (clones NL16, GOM2, ZG4 and RJ4, respectively, courtesy of Dr Margaret Goodall, University of Birmingham, UK). After 8 washes with PBS Tween-20 0.05%, antibodies belonging to IgG, IgA and IgM classes and IgG subclasses were revealed after 1 h incubation at 20° C. with peroxidase (HRP)-conjugated antisera (anti-mouse IgG for IgG subclasses, or anti-human IgG, IgA, and IgM, 1:5000 in washing buffer, Jackson ImmunoResearch Inc).

Washed plates were revealed with H₂O₂/o-phenylene-diamine substrate in urea buffer pH 5.0, the reaction stopped with 2N H₂SO₄, and optical densities (OD) measured at 492 nm.

Determination of Dilution Curve Parameters

Non-specific signals were subtracted from corresponding overall signals in order to retain values relating to specific binding of anti-Aβ antibodies. The best fitting curve for modeling serum dilutions followed a sigmoid model and was calculated using a non-linear least square approach to link specific OD with sample dilution as follows:

$$specific OD = \frac{a}{1 + b \cdot {e}^{- c . x}}$$

where x corresponds to the dilution factor expressed in logarithmic units. For a given curve, constants a, b and c were used in the following analysis.

The a constant represents the asymptotic maximum of the curve on the y-axis (OD units) that reflects the maximum amount of antigenic determinants bound by tested anti-Aβ antibodies. In order to obliterate its inter-assay variance, this parameter was expressed as a ratio with the maximum given by the curve of a reference serum pool included in all assays, i.e. serving as internal standard. Hence, what is hereafter termed Max has no unit and directly reflects the amount of coated binding sites, i.e. the epitopic diversity of anti-Aβ antibodies. The x-axis coordinate at the inflexion point of the sigmoid dilution curve can be calculated as ln(b)/c (where ln(b) is the napierian logarithm of b), and represents the dilution factor in logarithmic units that yields 50% of the Max signal. For a monoclonal antibody, this parameter depends on its concentration and on the affinity of the epitope-binding site. For polyclonal antibodies, this parameter reflects partially the overall concentration of anti-Aβ antibodies of a given isotype, and partially their overall avidity. This parameter will be hereafter termed Titer, and is expressed as an absolute number that represents a dilution factor in decimal logarithmic units. The steepness of the sigmoid curve at the inflexion point can be calculated as −c/4. It depends on the a constant and on cooperativity phenomena that can occur between distinct antibody binding sites. What will be hereafter termed Steepness corresponds to −c/4a and expresses the loss of relative OD units per dilution factor (in decimal logarithmic units), at the inflexion point of the curve.

A 4^{th} experimental parameter, hereafter referred to as K_{A}′ _{App}, corresponds to the apparent affinity of anti-Aβ antibodies of a given isotype. It was calculated through a linearization procedure of the sigmoid curve³ following the equation:

$$\frac{1}{1 - OD / a} = {K}_{A App}^{′} \times \frac{1 {0}^{- x}}{OD / a} + d$$

This parameter is expressed in dilution factor units and translates the overall avidity of anti-Aβ antibodies of a given isotype. Therefore, it is not independent from values of Steepness and Titer, because cooperativity phenomena influence apparent avidity, and apparent titer directly depends on avidity. However, these 3 parameters are not strictly equivalent, and each reflects in a subtle manner different aspects of antigen/antibody binding.

Determination of the MAST Index

A single index was calculated from the 4 parameters described above, yielded by analyses of each antibody isotype on each Aβ antigenic isoform. In order to reduce the number of variables of interest, and thus the family-wise error rate, we used the first principal component of PCA performed on overall parameters to summarize the maximum information given by the 4 redundant variables of each curve. It is worth noting that these coefficients did not differ significantly between clinical groups, antigenic preparations or antibody isotypes. The first component of PCA determined the weight coefficients of the index formula, as follows: Index=0.2316*Max+0.6717*Titer+0.1413*Steepness+0.6894*K′A App.

Quality Management of Multiplex ELISA

In order to minimize bias due to manipulator and inter-assay variability, samples were randomized and analyzed blindly. Nevertheless, each experiment included samples from all clinical groups, in such a way that any experimental bias would comparably affect results of all groups. In each experiment, an internal standard made of pooled human sera was used in order to normalize OD between experiments, standardize Max results, and thus reduce inter-assay variability due for instance to enzymatic revelation. Another pool of human sera served as an internal control in order to assess variability between experiments. ELISA plates that gave internal control results over 2.5×standard deviation (sd) from mean values on one or more parameters were discarded. Goodness-of-fit of the sigmoid curve modeling was assessed by square-deviation, and only assays with R>0.9 were validated.

Sigmoid Dilution Curve Parameters and Definition of a Summary Index

The best fitting curve for modeling serum dilutions followed a sigmoid model which, in addition with a linearization procedure, led to the measurement of 4 experimental parameters per curve: the maximum, the titer, the steepness, and the apparent avidity constant (K′_{A App}), which account for the number of bound epitopes, the serum concentration, and the overall apparent binding strength of the revealed antibody isotype. Besides inter-individual variability of naturally occurring antibodies, these parameters are not independent, reflecting different aspects of the anti-Aβ humoral immune response, such as clonal expansion and affinity maturation. Since they provide redundant information, we defined an index called MAST for Maximum, Avidity, Steepness, Titer, as a linear combination of the 4 parameters, weighted using first component factors from principal component analysis (PCA). This summary index quantifies concomitant variations in anti-Aβ antibodies diversity, concentration and avidity. It was determined for the 6 studied isotypes (IgG, IgA, IgM, IgG1, IgG3 and IgG4; IgG2 assays yielded insignificant signals in all tested cases) detected on 4 different antigenic preparations (s40, s42, f40 and f42 isoforms of Aβ).

Statistical Analysis

Differences in anti-Aβ antibodies indexes between CAA, CAA-he or CAA-ri patients and controls were assessed using Mann-Whitney test with Bonferroni correction procedure for multiple comparison (two-sided, α=0.05). Exploratory analysis of the raw dilution curve parameters affecting the index was performed post-hoc using one-sided tests, according to the sign of the corresponding coefficient within the index formula. The performances of the statistically significant indexes were assessed using ANOVA to test univariate logistic regression models against the null model, and using receiver operating characteristic (ROC) curves to determine overall sensitivity, specificity and area under the curve (AUCs). Optimal cutoff values were selected as corresponding to maximum accuracy (i.e. 0.5*(sensitivity+specificity). We limited our analysis to univariate models to avoid the selection of over-fitted models, and the robustness of each selected model was assessed by 10-fold stratified cross-validation procedure, with a critical lower limit of the 95% confidence interval below 50% of specificity, sensitivity or AUC. R version 3.3.2 was used for analyses (R Core Team, 2016). The ROCR package was used for performances analysis (Sing et al., 2005).

Results

The study included a first cohort (hereafter termed “discovery cohort”) of 60 subjects whose main characteristics are presented in Table 1 above. Nineteen out of the 20 patients with CAA-he met the diagnosis criteria for probable CAA with LH. The remaining patient had 2 thalamic MB but he also had more than 200 lobar MB along with typical amyloid spells (transient aphasia and visual flashes) and was therefore included in the CAA-he group. The other 19 patients had probable CAA with LH, and were studied in the acute setting of the LH. All CAA-ri patients but one (patient 5), met the diagnostic criteria of probable CAA-ri. In patient 5, although MRI diagnostic criteria were not met due to the absence of typical white matter hyperintensities (WMH), diagnosis was made upon cerebral biopsy. When the patient relapsed 10 months after treatment cessation, typical WMH and growth of MB count were typical of CAA-ri. Serum samples were stored during 19, 16, and 27 months (medians), respectively for the control, CAA-he and CAA-ri groups.

A second cohort (hereafter termed “replication cohort”) recruited 48 subjects according to the same inclusion criteria: 28 CAA-he patients, 8 CAA-ri patients, and 12 age-matched controls.

The reliability of the multiplex ELISA was assessed considering the goodness-of-fit of the sigmoid modeling, and the inter-assay variability, and showed performances within the acceptability limits. A single index was calculated from the parameters of each sigmoid dilution curve.

We expected correlations to be found between the four parameters of the dilution curves, as they reflect different aspects of antibodies (concentration, avidity and diversity of epitope recognition) that vary together during humoral responses. As expected, all 4 parameters were same-signed in the first-component, which implies positive correlation between the first component index and each raw parameter. Of note, the 4 coefficients associated to corresponding parameters for building the first component were within the same order of magnitude, i.e. below 5-fold fluctuation. This means that the index is not drastically over-determined by one parameter alone. The first-component explains over 80% of the variance, and hence translates efficiently into one index the overall variability of the 4 dilution curve parameters.

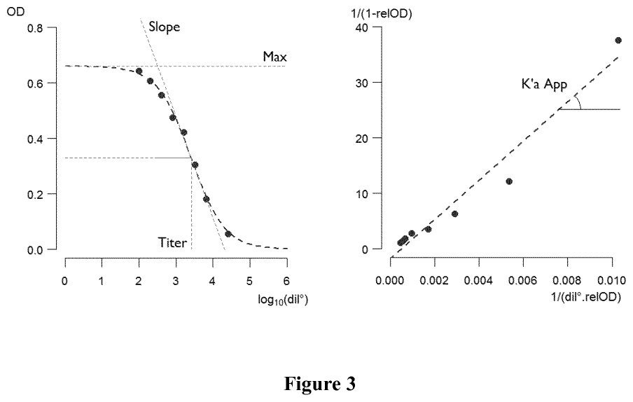

See FIG. 3 and Table 2.

TABLE 2
Sigmoid modeling goodness-of-fit and internal control mean CV, by antigenic
preparations and antibody isotypes.

| | Overall | s40 | s42 | f40 | f42 | IgG | IgA | IgM | IgG1 | IgG3 | IgG4 |
|---|---|---|---|---|---|---|---|---|---|---|---|
| R² (mean) | 0.97 | 0.98 | 0.98 | 0.97 | 0.97 | 0.99 | 0.98 | 0.99 | 0.98 | 0.96 | 0.95 |
| Max (mean CV) | 16% | 18% | 15% | 18% | 15% | 13% | 17% | 17% | 16% | 19% | 16% |
| Titre (mean CV) | 7% | 8% | 6% | 9% | 7% | 5% | 8% | 7% | 6% | 8% | 13% |
| Steepness (mean | 16% | 16% | 12% | 17% | 19% | 14% | 17% | 17% | 13% | 18% | 19% |
| CV) | | | | | | | | | | | |
| K'A App (mean | 12% | 9% | 9% | 15% | 14% | 8% | 13% | 15% | 11% | 12% | 10% |
| CV) | | | | | | | | | | | |

Goodness-of-fit was evaluated by mean R² with an arbitrary acceptance limit of R = 0.9. Reproducibility of each parameter was expressed with mean coefficient of variation (CV) for the corresponding parameter. Mean R² were obtained from dilution curves of patients, internal standard and internal controls (n = 2160, 540, and 360 for overall, by antigen, and by isotype R², respectively). Mean CV were obtained from dilution curves obtained with pooled sera serving as internal control (n = 360, 90 and 60 for overall, by antigen, and by isotype CV, respectively).

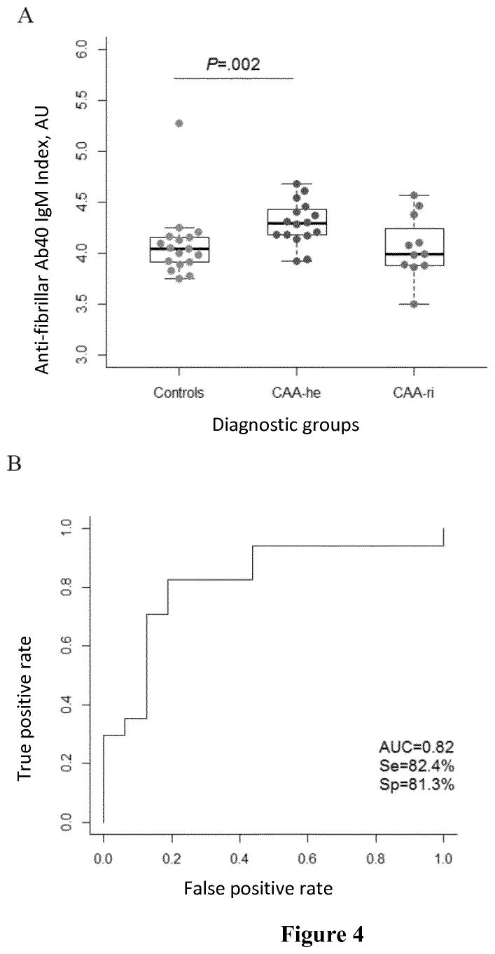

The anti-f40 IgM MAST-index was higher in overall CAA (median [IQR], 4.18 [3.99-4.39]; P=0.04) and in CAA-he (median [IQR], 4.29 [4.18-4.42]; P=0.002) compared with the control group (median [IQR], 4.04 [3.91-4.15]). No statistically significant difference appeared for CAA-ri patients compared with the control group (median [IQR], 3.99 [3.88-4.24]; P=0.96) (see FIG. 4A). Post-hoc analysis of the raw dilution curve parameters suggested higher titer (median [IQR], 2.82 [2.78-2.97] vs 2.71 [2.68-2.80]; P=0.02) and K′A App (median [IQR], 3.00 [2.82-3.06] vs 2.76 [2.70-2.95]; P=0.006) of anti-f40 IgM antibodies in CAA-he compared with controls, suggesting higher serum concentration, and higher avidity.

To evaluate the performances of anti-f40 IgM MAST-indexes as a potential biomarker of CAA-he, respectively, we performed logistic regression and ROC curve analyses. The univariate models using anti-f40 IgM performed significantly better against the null model to discriminate CAA-he (P=0.03) An anti-f40 IgM MAST-index cutoff of 0.5239 provided the optimal discrimination between patients CAA-he and controls at a sensitivity of 82.4% (95% CI, 61.5%-98.5%) and a specificity of 81.3% (95% CI, 51.5%-88.5%), with an AUC of 0.82 (95% CI, 0.53-0.82) (FIG. 4B).

When comparing CAA-he patients to age-matched controls and CAA-ri patients to age-matched controls, higher MAST-Index were found in the discovery cohort and confirmed in the replication cohort regarding the two diseased groups for anti-s40 IgG3 antibodies and anti-s40 IgG4 antibodies.

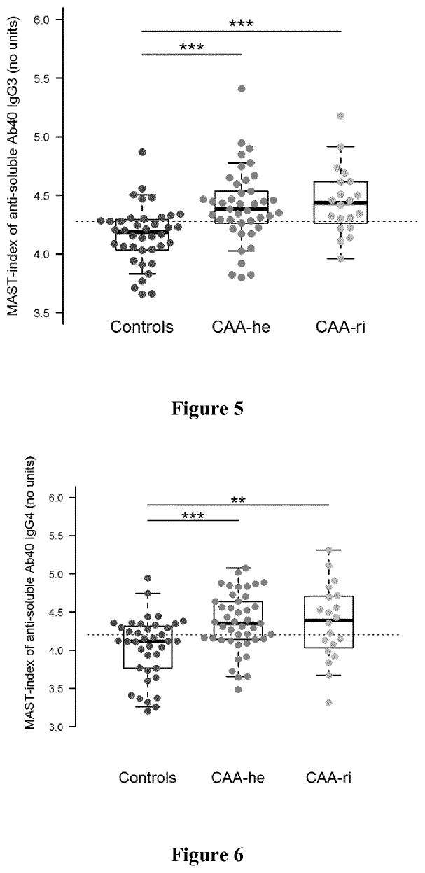

Anti-s40 IgG3 antibodies MAST-Index in CAA-he patients was higher in the discovery cohort (4.34 [4.11-4.43] as compared to 4.11 [3.87-4.22] in the control group; P=0.04). This result was confirmed in the replication cohort (4.45 [4.29-4.65] Index score in CAA-he patients as compared with 4.26 [4.07-4.31] in controls; P=0.006). A similar result was observed regarding CAA-ri patients: in the discovery cohort, the anti-s40 IgG3 antibodies MAST-Index was higher in CAA-ri patients (4.46 [4.33-4.56]; P<0.001). This was confirmed in the replication cohort (4.37 [4.26-4.63]; P<0.05) (FIG. 5).

Similarly to IgG3, anti-s40 IgG4 antibodies MAST-Index in CAA-he patients was higher in the discovery cohort (4.24 [4.09-4.35] as compared to 4.04 [3.63-4.29] in the control group; P<0.05). This result was confirmed in the replication cohort (4.49 [4.16-4.73] Index score in CAA-he patients as compared with 4.15 [4.11-4.34] in controls; P=0.01). Regarding CAA-ri patients, anti-s40 IgG4 antibodies MAST-Index was higher as compared to controls in the discovery cohort (4.14 [3.90-4.44]) and in the replication cohort (4.70 [4.49-4.96]; P=0.001) (FIG. 6).

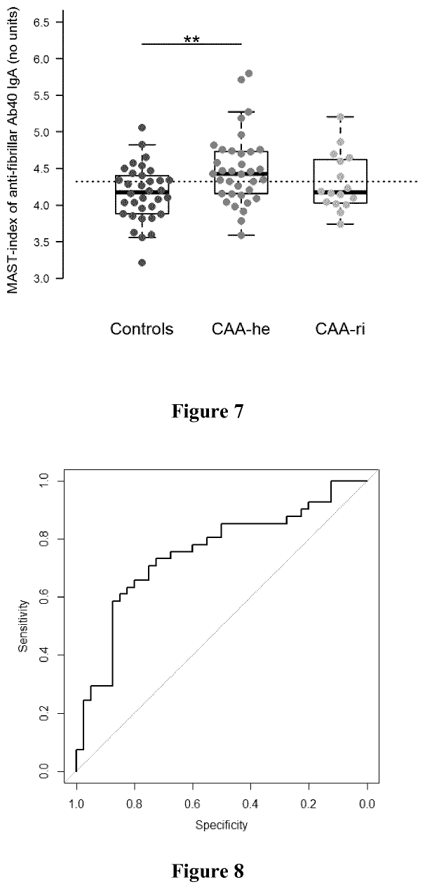

The MAST-Index of anti-f40 IgA was increased in CAA-he patients as compared to controls (4.44 [4.05-4.57] vs 4.14 [3.88-4.44]; P<0.05) in the discovery cohort. This was confirmed in the replication cohort (4.42 [4.20-4.78] vs 4.18 [4.10-4.26]; P=0.02) (FIG. 7).

In order to evaluate these parameters as a potential biomarker of CAA-related acute events, we carried out univariate logistic regression analyses by pooling the results of the two cohorts. The anti-s40 IgG3 MAST index performed significantly better against the null model to discriminate patients with CAA-he (P=0.002) or CAA-ri (P=0.001). In a same fashion, the anti-s40 IgG4 MAST index performed significantly better against the null model to discriminate patients with CAA-he (P=0.001) or CAA-ri (P=0.02). The anti-f40 IgA MAST index performed significantly better against the null model to discriminate patients with CAA-he (P=0.007) from controls.

To assess the performances of these MAST-indexes as biomarkers, ROC curve and diagnostic characteristics were computed.

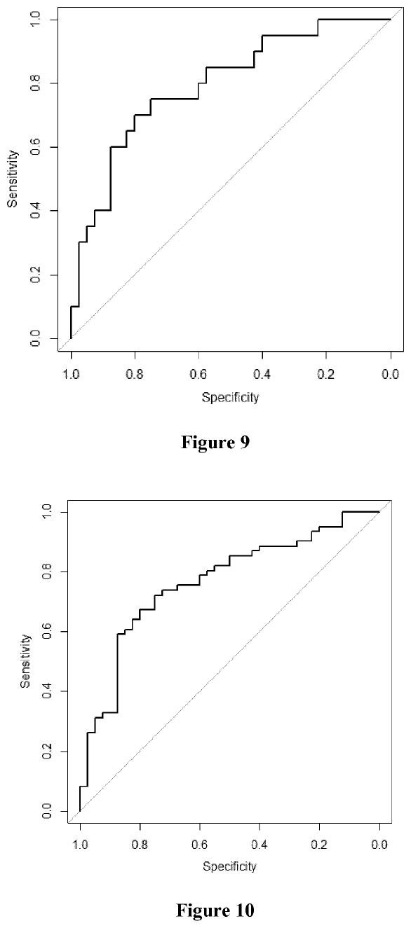

For the anti-s40 IgG3 MAST-index, the area under the ROC curve (AUC) was 0.75 [0.64-0.86] for CAA-he patients against controls (FIG. 8), 0.79 [0.67-0.92] for CAA-ri patients against controls (FIG. 9), and 0.77 [0.67-0.86] for CAA patients with acute events against controls (FIG. 10). The anti-s40 IgG3 MAST-index had 72.5% sensitivity (95% CI, 62-81%) and 73% specificity (95% CI, 63-82%) for discriminating CAA-he patients from controls at the threshold of 4.279, 80% sensitivity (95% CI, 68-88%) and 70% specificity (95% CI, 57-80%) for discriminating CAA-ri patients from controls at the threshold of 4.307, and 75% sensitivity (95% CI, 66-82%) and 72% specificity (95% CI, 63-80%) for discriminating CAA patients with acute events from controls, at the threshold of 4.279.

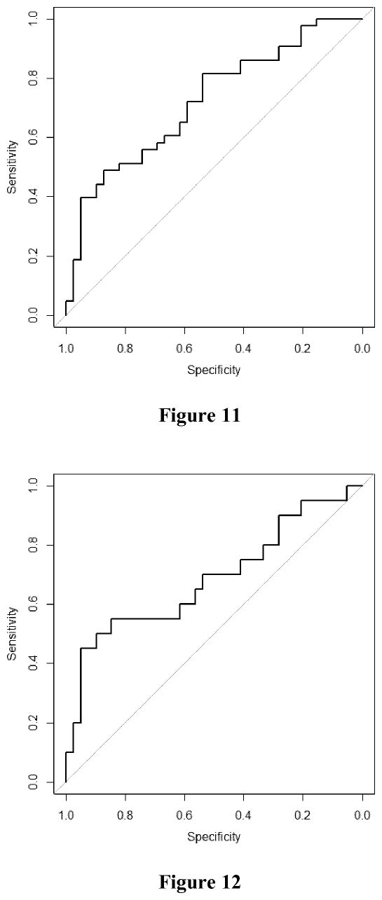

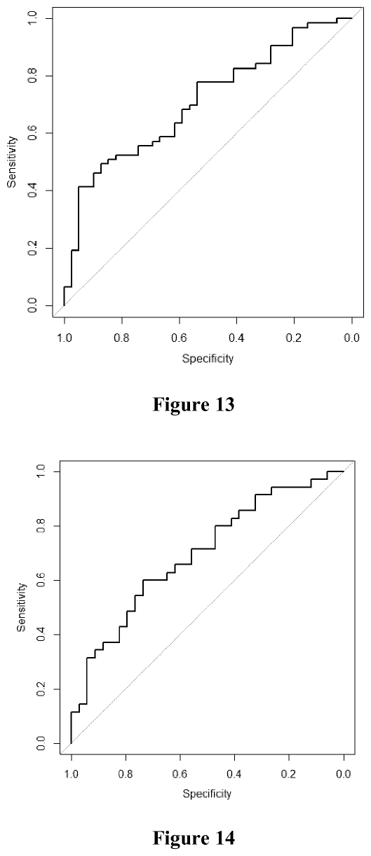

For the anti-s40 IgG4 MAST-index, the AUC was 0.72 [0.61-0.83] for CAA-he patients against controls (FIG. 11), 0.69 [0.53-0.84] for CAA-ri patients against controls (FIG. 12), and 0.71 [0.61-0.81] for CAA patients with acute events against controls (FIG. 13). The anti-s40 IgG4 MAST-index had 62% sensitivity (95% CI, 51-71%) and 63% specificity (95% CI, 52-72%) for discriminating CAA-he patients from controls at the threshold of 4.205, 61% sensitivity (95% CI, 49-73%) and 60% specificity (95% CI, 47-72%) for discriminating CAA-ri patients from controls at the threshold of 4.201, and 62% sensitivity (95% CI, 52-70%) and 63% specificity (95% CI, 54-72%) for discriminating CAA patients with acute events from controls, at the threshold of 4.202.

For the anti-f40 IgA MAST-index, the AUC was 0.695 [0.57-0.82] for CAA-he patients against controls (FIG. 14). The anti-f40 IgA MAST-index had 65% sensitivity (95% CI, 53-75%) and 63% specificity (95% CI, 51-73%) for discriminating CAA-he patients from controls, at the threshold of 4.322.

### CONCLUSION

This case-control study demonstrates an association between serum anti-Aβ antibody features and subtypes of CAA manifestations. Higher MAST-index, titer, avidity and diversity of circulating anti-soluble Aβ₁₋₄₀ IgG3 or IgG4 antibodies were associated with CAA-he and CAA-ri, while higher MAST-index, titer and avidity circulating anti-fibrillar Aβ₁₋₄₀ IgM or IgA were associated with CAA-he alone.

### REFERENCES

Auriel E, Charidimou A, Gurol M E, et al. Validation of Clinicoradiological Criteria for the Diagnosis of Cerebral Amyloid Angiopathy-Related Inflammation. JAMA Neurol. 2016; 73(2):197-202.
Béjot Y, Cordonnier C, Durier J, Aboa-Eboulé C, Rouaud O, Giroud M. Intracerebral haemorrhage profiles are changing: results from the Dijon population-based study. Brain. 2013; 136(2):658-64.
Chantran Y, Capron J, Alamowitch S, Aucouturier P. Anti-Aβ_Antibodies and Cerebral Amyloid Angiopathy Complications. Front Immunol. 2019 10:1534.
Charidimou A, Boulouis G, Gurol M E, et al. Emerging concepts in sporadic cerebral amyloid angiopathy. Brain. 2017; 140(7):1829-1850.
Fazekas F, Chawluk J B, Alavi A, Hurtig H I, Zimmerman R A. M R signal abnormalities at 1.5 T in Alzheimer's dementia and normal aging. AJR Am J Roentgenol. 1987; 149(2):351-6.
Gregoire S M, Chaudhary U J, Brown M M, et al. The Microbleed Anatomical Rating Scale (MARS): reliability of a tool to map brain microbleeds. Neurology. 2009; 73(21):1759-66.
Knudsen K A, Rosand J, Karluk D, Greenberg S M. Clinical diagnosis of cerebral amyloid angiopathy: validation of the Boston criteria. Neurology. 2001; 56(4):537-9.
Linn J, Halpin A, Demaerel P, et al. Prevalence of superficial siderosis in patients with cerebral amyloid angiopathy. Neurology. 2010; 74(17):1346-50.
Martinez-Ramirez S, Romero J R, Shoamanesh A, et al. Diagnostic value of lobar microbleeds in individuals without intracerebral hemorrhage. Alzheimers Dement. 2015; 11(12):1480-1488.
R Core Team (2016). R: A language and environment for statistical computing. R Foundation for Statistical Computing, Vienna, Austria. https://www.R-project.org/.
Salvarani C, Hunder G G, Morris J M, Brown R D Jr, Christianson T, Giannini C. Aβ-related angiitis: comparison with CAA without inflammation and primary CNS vasculitis. Neurology. 2013; 81(18):1596-603.
Scheltens P, Leys D, Barkhof F, et al. Atrophy of medial temporal lobes on MRI in “probable” Alzheimer's disease and normal ageing: diagnostic value and neuropsychological correlates. J Neurol Neurosurg Psychiatry. 1992; 55 (10): 967-72.
Sing T, Sander O, Beerenwinkel N and Lengauer T (2005). “ROCR:visualizing classifier performance in R.” _Bioinformatics_, *21*(20), pp. 7881. http://rocr.bioinf.mpi-sb.mpg.de.
Sperling R A, Jack C R Jr, Black S E, et al. Amyloid-related imaging abnormalities in amyloid-modifying therapeutic trials: recommendations from the Alzheimer's Association Research Roundtable Workgroup. Alzheimers Dement. 2011; 7(4):367-85.
Stine et al. Preparing Synthetic Aβ in Different Aggregation States. Methods Mol Biol. 2011; 670: 13-32.
Wilson, Ambler, Shakeshaft, et al. Cerebral microbleeds and intracranial haemorrhage risk in patients anticoagulated for atrial fibrillation after acute ischaemic stroke or transient ischaemic attack (CROMIS-2): a multicentre observational cohort study. Lancet Neurol. 2018; 17(6):539-47
Yamada M. Cerebral amyloid angiopathy: emerging concepts. J Stroke. 2015; 17(1):17-30.

1. An in vitro method for diagnosing cerebral amyloid angiopathy (CAA), or for diagnosing or determining the risk of developing a complication of CAA, in a human subject,
which method comprises analyzing serial dilutions of a plasma or serum sample of the subject, for determining at least one binding parameter of antibodies present therein, wherein said antibodies are anti-Ab amyloid peptide(s) antibodies, which method comprises detecting said antibodies by means of a chromogenic or fluorescent labelling, preferably wherein the antibodies are detected by means of an indirect immunoassay on immobilized antigen using a secondary anti-human immunoglobulin antibody that carries a chromogenic enzyme or fluorescent label;
and wherein the method comprises determining an index (designated “MAST-index”) that is a weighted summation of the following binding parameters: i) the titer of said antibodies as determined at 50% maximum binding, ii) the affinity or avidity constant, iii) the maximum optical density, preferably normalized with an internal standard, and optionally iv) the steepness of the dilution curve.

2. The method of claim 1, wherein the MAST index is defined as a1×(the maximum optical density observed)+a2×(the affinity or avidity constant)+a3×(the steepness of the dilution curve)+a4×(the titer of said antibodies as determined at 50% of maximum binding), wherein coefficients a1, a2, a3, a4 are as follows:
a1 is 5 to 30% of the sum of all coefficients (a1+a2+a3+a4)
a2 is 25 to 50% of the sum of all coefficients (a1+a2+a3+a4)
a3 is 0 to 20% of the sum of all coefficients (a1+a2+a3+a4)
a4 is 25 to 50% of the sum of all coefficients (a1+a2+a3+a4).

3. The method of claim 1, wherein the complication is hemorrhagic CAA or inflammatory CAA.

4. The method of claim 1, wherein the Ab peptide is Ab₁₋₄₀ or Ab₁₋₄₂ peptide.

5. The method of claim 4, wherein the Ab peptide is soluble Ab₁₋₄₀.

6. The method of claim 5, wherein the anti-soluble Ab₁₋₄₀ antibodies are IgG antibodies, preferably IgG3, IgG1 or IgG4 antibodies, still preferably IgG3 or IgG4 antibodies.

7. The method of claim 4, wherein the Ab peptide is fibrillar Ab₁₋₄₀.

8. The method of claim 7, wherein the anti-fibrillar Ab₁₋₄₀ antibodies are IgM antibodies.

9. The method of claim 1, comprising determining the MAST-index of anti-soluble Ab₁₋₄₀ IgG antibodies, wherein a higher MAST-index of anti-soluble Ab₁₋₄₀ IgG antibodies compared to control is indicative of a subject with a CAA complication of the inflammatory (CAA-ri) type, or of a higher risk for the subject to develop a CAA-ri, preferably wherein the anti-soluble Ab₁₋₄₀ IgG antibodies are anti-soluble Ab₁₋₄₀ IgG3 or IgG4 antibodies.

10. The method of claim 1, comprising determining the MAST-index of anti-fibrillar Ab₁₋₄₀ IgM or IgA antibodies, wherein a higher MAST-index of anti-fibrillar Ab₁₋₄₀ IgM or IgA antibodies compared to control is indicative of a subject with a CAA complication of the haemorrhagic (CAA-he) type, or of a higher risk for the subject to develop a CAA-he.

11. The method of claim 1, which comprises determining at least one binding parameter of anti-soluble Ab₁₋₄₀ IgG antibodies and at least one binding parameter of anti-fibrillar Ab₁₋₄₀ IgM or IgA antibodies in a plasma or serum sample of the subject.

12. The method of claim 11, which comprises conducting a multiplex immunoassay.

13. An in vitro method for diagnosing cerebral amyloid angiopathy (CAA), or for diagnosing or determining the risk of developing a complication of CAA, especially a CAA complication of the inflammatory (CAA-ri) type, in a human subject, which method comprises measuring the anti-soluble Ab₁₋₄₀ IgG3 or IgG4 antibodies.

14. The method of claim 1, wherein the subject is afflicted with Alzheimer's disease, cortical posterior atrophy, primary progressive aphasia, or Down's syndrome.

15. The method of claim 2, wherein the Ab peptide is Ab₁₋₄₀ or Ab₁₋₄₂ peptide.

16. The method of claim 3, wherein the Ab peptide is Ab₁₋₄₀ or Ab₁₋₄₂ peptide.

17. The method of claim 15, wherein the Ab peptide is soluble Ab₁₋₄₀.

18. The method of claim 16, wherein the Ab peptide is soluble Ab₁₋₄₀.

19. The method of claim 17, wherein the anti-soluble Ab₁₋₄₀ antibodies are IgG antibodies, preferably IgG3, IgG1 or IgG4 antibodies, still preferably IgG3 or IgG4 antibodies.

20. The method of claim 18, wherein the anti-soluble Ab₁₋₄₀ antibodies are IgG antibodies, preferably IgG3, IgG1 or IgG4 antibodies, still preferably IgG3 or IgG4 antibodies.
